(19) United States
(12) Patent Application Publication
(10) Pub. No.: US 2004/0170963 A1
(43) Pub. Date: Sep. 2, 2004

(54) Antivirus RNA

(76) Inventors: Ih-Jen Su, Taipei City (TW); Wen-Tsan Chang, Tainan (TW); Huan-Yao Lei, Tainan (TW); Tsung-Lin Cheng, Tainan (TW); Wen-Wei Chang, Tainan (TW); Ming-Derg Lai, Tainan (TW); Wen-Ya Huang, Tainan (TW)

Correspondence Address: FISH & RICHARDSON PC, 225 FRANKLIN ST, BOSTON, MA, 02110, US

(21) Appl. No.: 10/727,355

(22) Filed: Dec. 3, 2003

Related U.S. Application Data
(60) Provisional application No. 60/430,719, filed on Dec. 3, 2002.

(51) Int. Cl. C12Q 1/70; C07H 21/02; A61K 48/00

(52) U.S. Cl. 435/5; 514/44; 536/23.1

(57) ABSTRACT

An RNA molecule for inhibiting expression of a gene of a virus. Also disclosed are a DNA vector encoding the RNA molecule and a pharmaceutical composition containing the RNA molecule or vector and a pharmaceutically acceptable carrier, as well as methods for inhibiting expression of a gene of a virus, replication of the virus, and infection with the virus.

### RELATED APPLICATION

This application claims priority to U.S. Provisional Application Serial No. 60/430,719, filed Dec. 3, 2002, the contents of which are incorporated herein by reference.

### BACKGROUND

Virus is the cause of various disorders, including hepatitis. Viral hepatitis is the single most important cause of liver diseases. Take hepatitis B for example. It is estimated to affect over 300 million people worldwide. Patients with liver damage resulting from hepatitis B may develop chronic liver diseases, such as cirrhosis and hepatocellular carcinoma. It can be treated by interferon. However, only a fraction of hepatitis B patients are responsive to the treatment. Other drawbacks to interferon therapy include significant side effects and high costs. New drugs for treating hepatitis B have been developed. Nonetheless, many of them tend to induce resistance. Thus, there is a need for an effective drug for treating viral infection, such as hepatitis B and other viral hepatitis.

### SUMMARY

This invention is based, at least in part, on the discovery that an RNA molecule corresponding to a segment of hepatitis B virus (HBV) gene encoding the Hepatitis B Surface antigen (HBsAg) inhibits not only the expression of HBsAg but also the replication of HBV.

Accordingly, in one aspect, this invention features an RNA for inhibiting expression of a gene of a virus, such as that of a hepatitis virus (e.g., HBV). The RNA contains a first nucleotide sequence that hybridizes under stringent conditions to a segment of the gene, and a second nucleotide sequence that is complementary to the first nucleotide sequence and hybridizes to the first nucleotide sequence to form a duplex structure. The term “stringent conditions” refers to conditions for hybridization in 6× sodium chloride/sodium citrate (SSC) at about 45° C., followed by one or more washes in 0.2×SSC, 0.1% SDS at 50-65° C. In one embodiment, the first nucleotide sequence and the second nucleotide sequence are on the same strand. In another embodiment, they are on two different strands to form a double-stranded RNA. For efficient inhibition of the expression of a viral gene, the first nucleotide sequence is at least 19, e.g., 19-29, nucleotides in length. To inhibit the expression of HBsAg, the first nucleotide sequence is designed to hybridize under stringent conditions to a segment containing one of SEQ ID NOs: 1-10 (shown below). In one embodiment, the first nucleotide sequence is the same as or complementary to one of these sequences, e.g., SEQ ID NO: 3.

| HBsAg-1: | 5′-CCTGCACGACTCCTGCTCA-3′ | (SEQ ID NO: 1) |
|---|---|---|
| HBsAg-2: | 5′-TACCTATGGGAGTGGGCCT-3′ | (SEQ ID NO: 2) |
| HBsAg-3: | 5′-GGTATGTTGCCCGTTTGTC-3′ | (SEQ ID NO: 3) |
| HBsAg-4: | 5′-GGCAGCCTACTCCCATCTC-3′ | (SEQ ID NO: 4) |
| HBsAg-5: | 5′-GAGACAGTCATCCTCAGGC-3′ | (SEQ ID NO: 5) |
| HBsAg-6: | 5′-TCTCCGCGAGGACTGGGGA-3′ | (SEQ ID NO: 6) |
| HBsAg-7: | 5′-CAACCAGTACGGGACCATG-3′ | (SEQ ID NO: 7) |
| HBsAg-8: | 5′-GTCCTCTAATTCCAGGATC-3′ | (SEQ ID NO: 8) |
| HBsAg-9: | 5′-GCCTCATCTTCTTATTGGT-3′ | (SEQ ID NO: 9) |
| HBsAg- | 5′-CCTCCAATCACTCACCAAC-3′ | (SEQ ID NO: 10) |
| 10: | | |

The invention also features a DNA vector that contains a nucleic acid from which the above-described RNA is transcribed, i.e., encoding the RNA. A vector refers to a nucleic acid molecule capable of transporting another nucleic acid to which it has been linked. The vector is capable of autonomous replication or integrating into a host DNA. Examples of the vector include a plasmid, cosmid, or viral vector. The vector of this invention includes a nucleic acid in a form suitable for transcribing the nucleic acid in a host cell. Preferably, the vector includes one or more regulatory sequences operatively linked to the nucleic acid sequence to be transcribed. A “regulatory sequence” refers to a promoter, enhancer, and other expression control elements (e.g., polyadenylation signal). Regulatory sequences include those that direct constitutive transcription of a nucleotide sequence, as well as tissue-specific regulatory and/or inducible sequences. The design of the vector depends on considerations such as the choice of the host cell to be transformed, the level of transcription of RNA desired, and the like.

The invention further features a method of reducing the expression of a gene of a virus, such as that of a hepatitis virus, e.g., HBV, in a cell by introducing into the cell an effective amount of the above-described RNA or vector. This method can be used in inhibiting the replication of the virus in the cell. It can also be used in treating an infection with that virus, such as hepatitis B, by administering to a subject in need thereof an effective amount of the RNA or vector.

Finally, the invention features a pharmaceutical composition that contains the above-described RNA or vector, and a pharmaceutically acceptable carrier. This composition can be used in treating an infection with a virus.

The details of one or more embodiments of the invention are set forth in the accompanying description below. Other advantages, features, and objects of the invention will be apparent from the detailed description and the claims.

### DETAILED DESCRIPTION

This invention relates to RNAs and their use in preventing or treating an infection with virus, such as hepatitis.

For example, within the scope of this invention is an RNA that inhibits the expression of a viral gene via RAN interference. RNA interference (RNAi) is a process in which double-stranded RNA (dsRNA) directs homologous sequence-specific degradation of messenger RNA. In mammalian cells, RNAi can be triggered by 21-nucleotide duplexes of small interfering RNA (siRNA) without activating the host interferon response. As this process represses the expression of a viral gene in the cells, it can be used to treat an infection with a virus.

One can determine which viral gene to target based on whether a protein encoded by a viral gene plays roles in an infection or in the corresponding pathogenesis. In general, valid targets are genes required for viral replication, viron formation, binding to or entering target cells, or pathogenesis of the infection or the associated disorders.

Take HBsAg for an example. It is required for forming the envelope of HBV and mature virus particles. Further, study from HBsAg-transgenic mice shows that HBsAg plays roles in hepatitis and the development of associated disorders, e.g., hepatocellular carcinoma (HCC). Hepatocytes in some of these transgenic mice do not secrete HBsAg but store it in the endoplasmic reticulum, resulting in liver dysplasia at approximately 9 months of age, and oval-cell proliferation, nodule formation, and eventually, primary hepatocellular carcinoma at 15 to 18 months of age. HBsAg not only induces liver injury directly, but also acts as a co-carcinogen by altering detoxification of alfatoxin. Further, the above-mentioned hepatocellular damage leads to the compensating growth of hepatocytes. These proliferating hepatocytes, vulnerable to carcinogens and free radicals, are prone to mutation, thereby leading to hepatocarcinogenesis. Accordingly, HBsAg can be selected as a target for RNAi described herein.

Other example of targets include genes for protein synthesis, such as the 5′ most 1500 nucleotides of the HBV pregenomic mRNAs. See, e.g., Renbao et al., 1987, Sci. Sin. 30, 507. This region controls the translational expression of the core protein (C), X protein (X) and DNA polymerase (P) genes and plays a role in the replication of the viral DNA by serving as a template for reverse transcriptase. Disruption of this region in the corresponding RNA results in deficient protein synthesis as well as incomplete DNA synthesis. Targeting sequences 5′ of the encapsidation site can result in the inclusion of the disrupted 3′ RNA within the core virion structure and targeting sequences 3′ of the encapsidation site can result in the reduction in protein expression from both the 3′ and 5′ fragments.

An RNA of this invention can be synthesized by techniques well known in the art. See, e.g., Caruthers et al., 1992, Methods in Enzymology 211, 3-19, Wincott et al., 1995, Nucleic Acids Res. 23, 2677-2684, Wincott et al., 1997, Methods Mol. Bio. 74, 59, Brennan et al., 1998, Biotechnol Bioeng., 61, 33-45, and Brennan, U.S. Pat. No. 6,001,311. The RNA can also be transcribed from an expression vector and isolated using standard techniques.

An RNA or vector of this invention can be delivered to a virus target cells using method also well known in the art. See, e.g., Akhtar et al., 1992, Trends Cell Bio. 2, 139. For example, it can be introduced into cells using liposomes, hydrogels, cyclodextrins, biodegradable nanocapsules, or bioadhesive microspheres. Alternatively, the RNA or vector is locally delivered by direct injection or by use of an infusion pump. Other approaches include the use of various transport and carrier systems, for example though the use of conjugates and biodegradable polymers.

Also within the scope of this invention is a pharmaceutical composition that contains the above-descried RNA or vector as an active agent and a pharmaceutically acceptable carrier for treating an infection with a virus and a method of using such a composition in an effective amount to treat patents in need thereof. The term “treating” is defined as administration of a composition to a subject, who has a viral infection, with the purpose to cure, alleviate, relieve, remedy, prevent, or ameliorate the disorder, the symptom of the disorder, the disease state secondary to the disorder, or the predisposition toward the disorder. An “effective amount” is an amount of the composition that is capable of producing a medically desirable result, e.g., as described above, in a treated subject. An active agent can be formulated into dosage forms for different administration routes utilizing conventional methods. For example, it can be formulated in a capsule, a gel seal, or a tablet for oral administration. The pharmaceutical composition can also be administered via the parenteral route. The dosage required depends on the choice of the route of administration, the nature of the formulation, the nature of the subject's illness, the subject's size/weight/surface area/age/sex; and co-administration with other drugs. The efficacy of the pharmaceutical composition can be preliminarily evaluated in vitro. For in vivo studies, the composition can be injected into an animal and its therapeutic effects are then accessed.

The specific example below is to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

siRNA Vectors

Ten segments in the gene encoding HBsAg, HBsAg1-10 (SEQ ID NOs: 1-10), were selected as targets for RNA interference. To generate vectors that encode iRNAs targeting these segments, oligonucleotides containing each of these sequences (“sense stand”) and their inverted repeats (“anti-sense strand”) were synthesized using a standard method. In each of the oligonucleotides, the sense and antisense strands (“N₁₉”), 19 nucleotides in length, are separated by a spacer of 9 nucleotides. The complements of these oligonucleotides were also synthesized and allowed to form double-stranded DNAs with these oligonucleotides, respectively. An exemplary double-stranded DNA is shown below.

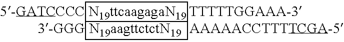

As shown above, each double-stranded DNA contained sequences flanking the N₁₉-spacer-N₁₉ segment (boxed), which created 5′ overhangs of Bgl II and Hind III at its two ends (underlined) to facilitate subsequent cloning. The double-stranded DNA was then ligated into a pSUPER vector (Dr. Reuven Agami, Plesmanlaan 121, 1066 cx Amsterdam, Netherlands) that had been digested with Bgl II and Hind III. The resultant vectors, once introduced in mammalian cells, transcribed hairpin RNAs. The pHsU6-+1 vector (Dr. Wen-Tsen Chang, Department of Biochemistry, NCKU Medical College, Tainan Taiwan 70101, ROC) was also used to construct vectors to generate hairpin RNAs. Shown below is an exemplary double-stranded DNA for cloning into this vector. It had 5′ overhangs of Cla I and Hind III.

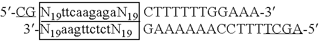

Inhibition of HBsAg Expression In Vitro

The effects of siRNA on HBsAg expression was examined in human hepatoma cell line Huh7 cells. Briefly, Huh7 cells were cotransfected with 1.5 μg of pSUPER or each of the above-described ten pSUPER-siRNA constructs, and 0.5 μg p(3A)SAg (Dr. C. C. Lu, Department of Pathology, Cheng Kung University, Taiwan) by Lipofectamine® 2000 (Invitrogen Corporation, Carlsbad, Calif. 92008). p(3A)SAg encodes the full length HBsAg protein.

Sixty hours later, whole-cell extracts were prepared from the transfected cells and the expression level of HBsAg was examined by enzyme-linked immunosorbent assay (ELISA, General Biologicals Corp., Taiwan). The results show that the HBsAg levels in the cells transfected with pSUPER-HBsAgs-1, -3, -4, -5, -6, -7, -9, ad -10 are lower than that in the cells transfected with pSUPER by 30% to 99%. In particular, the HBsAg expression level in the cells transfected with pSUPER-HBsAg-3 was inhibited by about 99%, and those in the cells transfected with pSUPER-HBsAgs-6, -7, -9, and -10 were also inhibited by more than 90%. These results indicated that the hairpin RNAs transcribed from most of the 10 pSUPER-HBsAg vectors functioned as iRNAs and inhibited the expression of HBsAg.

To examine the specificity of the iRNAs, pSUPER-HBsAg-3 and pSUPER were co-transfected into Huh7 cells, respectively, with pP_{sAg}-luc+, which encodes a luciferase, in the same manner described above. Sixty hours later, whole-cell extracts were prepared from the two transfected cell populations and analyzed for luciferase expression by a luciferase activity assay (Tropix, Inc., Bedford, Mass. 01730). The results show that the luciferase activities of the two cell populations are about the same, indicating that the RNA transcribed from pSUPER-HBsAg-3 specifically inhibits the expression of HBsAg but not that of other protein, such as luciferase.

The above-described experiments were repeated except that pHsU6-+1 and pHsU6-+1-HBsAg-3 were used instead of pSUPER and pSUPER-HBsAg-3. Similar results were obtained.

To study whether the sense RNA and anti-sense RNA corresponding to HBsAg-3 inhibited the expression of HBsAg, HBsAg-3 DNA was cloned into pHsU6-+1 in the sense and anti-sense orientations to generate pHsU6-+1-Sense-HBsAg-3 and pHsU6-+1-Anti-HBsAg-3, respectively. Each of these two vectors, a mixture of them, pHsU6-+1-HBsAg-3, and pHsU6-+1, were co-transfected with p(3A)SAg, respectively, into Huh7 cells, and examined for the expression of HBsAg in the same manner described above. The results show that neither pHsU6-+1-Sense -HBsAg-3 nor pHsU6-+1-Anti-HBsAg-3 significantly repressed the expression of HBsAg. The mixture of these two vectors repressed the expression by about 70%, which is lower than that exerted by pHsU6-+1-HBsAg-3 (99%). These results indicate that double-stranded RNA is required for inhibiting the expression of HBsAg.

Inhibition of Replication of HBV Zenome In Vitro

The effects of the above-described 10 pSUPER-HBsAg vectors on the whole HBV genome were examined. The 10 vectors were co-transfected in to Huh7 cells, respectively, with pHBV 3.6 (provided by Dr. L. P. Ting, Department of Microbiology & Immunology, Yang-Ming University), which contains all HBV open reading frames (ORFs). The expression level of HBsAg, as a proxy of the whole HBV genome level, was examined by ELISA in the manner described above. It was found that the HBsAg levels were repressed in the cells transfected with pSUPER-HBsAgs-3, -4, -5, -6, -7, -9, or -10.

The levels of the pregenomic and mature HBsAg mRNAs were examined by Northern blot. Thirty micrograms of total RNA was purified from the transfected Huh7 cells, separated by electrophoresis, and transferred onto a nylon membrane. The membrane was hybridized with a ³²P-labeled specific DNA probe, which corresponds to nt.181 to nt.895 of HBV adw2 subtype, at 56° C. overnight. The pregenomic and mature HBsAg mRNAs were visualized by autoradiography. Further, the HBsAg protein level in the transfected cells was studied by standard immunocytochemistry using a sheep polyclonal anti-HBsAg primary antibody (Serotec, Oxford, UK) and a biotinylated anti-sheep secondary antibody. The protein was visualized by peroxidase-conjugated streptoavidin (DAKO, Carpinteria, Calif.) and aminoethyl carbazole (AEC, Zymed Laboratories, San Francisco, Calif.). The results obtained from the Northern blot and immunocytochemistry analyses were consistent with those obtained from the above-described ELISA analysis.

Inhibition of HBsAg Expression and HBV Replication In Vivo

Thirty BALB/c mice, 8-12 week-old and weighing 20-25 g, were purchased from the Jackson Laboratory (Bar Harbor, Me.) or Charles River Japan, Inc. (Atsugi, Japan). The mice were divided into 10 groups, three in each group. The above-described pSUPER-HBsAg and p(3A)SAg vectors were dissolved in Ringers' solution (NaCl 0.154 M, KCl 5.63 mM, CaCl₂ 2.25 mM). The resultant mixture was injected into each mouse through the tail vein within 7 seconds at a dose of 2.2 ml/20 g body weight. Via hydrodynamics-based transfection, the DNA vectors were introduced into each mouse's hepatocytes.

At days 1-5 after the injection, serum was collected from each of the mice. The serum HBsAg levels were determined by ELISA (General Biological Corp., Taiwan, R.O.C.). The results are summarized below in Table 1.

TABLE 1
Effects of pSUPER-HBsAg vectors on serum HBsAg levels (ng/ml)

| p(3A)SAg + | Day 1 | Day 2 | Day 3 | Day 4 | Day 5 |
|---|---|---|---|---|---|
| PSUPER | 191.76 ± 34.45 | 459.20 ± 57.05 | 628.00 ± 119.90 | 583.70 ± 55.60 | 454.30 ± 95.74 |
| pSUPER-HBsAg-1 | 68.02 ± 12.79 | 573.90 ± 142.00 | 689.20 ± 72.45 | 522.20 ± 71.55 | 432.50 ± 111.70 |
| pSUPER-HBsAg-2 | 51.55 ± 34.81 | 584.40 ± 140.20 | 623.10 ± 190.80 | 451.30 ± 239.60 | 299.80 ± 198.30 |
| pSUPER-HBsAg-3 | 0 | 0 | 0 | 0 | 0 |
| pSUPER-HBsAg-4 | 21.65 ± 3.21 | 273.60 ± 49.45 | 267.30 ± 105.40 | 231.60 ± 60.81 | 248.35 ± 35.77 |
| pSUPER-HBsAg-5 | 43.39 ± 8.61 | 302.40 ± 116.10 | 276.60 ± 152.40 | 218.50 ± 99.46 | 234.90 ± 52.06 |
| pSUPER-HBsAg-6 | 47.04 ± 20.25 | 236.70 ± 107.50 | 198.70 ± 29.92 | 194.70 ± 65.89 | 188.60 ± 106.20 |
| pSUPER-HBsAg-7 | 37.98 ± 8.95 | 114.20 ± 14.07 | 90.77 ± 16.28 | 66.98 ± 22.55 | 69.01 ± 19.90 |
| pSUPER-HBsAg-8 | 30.95 ± 10.82 | 136.90 ± 47.44 | 78.42 ± 38.97 | 23.69 ± 1.31 | 3.05 ± 8.41 |
| pSUPER-HBsAg-9 | 132.10 ± 28.68 | 270.10 ± 43.93 | 234.30 ± 110.00 | 226.70 ± 59.27 | 168.40 ± 36.08 |

As shown in Table 1, in the absence of a pSUPER-HBsAg vector, the serum HBsAg level peaked at day 3 after the injection. pSUPER-HBsAg-1 or pSUPER-HBsAg-2 did not reduce the serum HBsAg level significantly. In contrast, pSUPER-HBsAg-3 inhibited the serum HBsAg level completely. In addition, pSUPER-HBsAg-7 or pSUPER-HBsAg-8 inhibited the serum HBsAg level by 70-80%, whereas pSUPER-HBsAg-4, pSUPER-HBsAg-5, pSUPER-HBsAg-6, or pSUPER-HBsAg-9 only partially inhibited the serum HBsAg level.

The inhibition of HBsAg production by pSUPER-HBsAg-3 was pSUPER-HBsAg to p(3A)SAg ratio-dependent. When the ratio was 5:1 (10 μg: 2 μg), the inhibition was almost 100%. When it was 2:1 or 1:1, the level of inhibition was gradually decreased. See Table 2 below.

TABLE 2
Ratio-dependent inhibition of serum HBsAg levels (ng/ml)

| Treatment^{a} | HBsAg level | | | | |
|---|---|---|---|---|---|
| Ratio | Day 1 | Day 2 | Day 3 | Day 4 | Day 5 |
| pSUPER(10 μg) + p(3A) | 191.8 ± 34.5 | 459.2 ± 57.1 | 628.0 ± 119.9 | 583.7 ± 55.6 | 454.3 ± 95.7 |
| SAg (2 μg) | | | | | |
| pSUPER-HBsAg-3(10 μg) + p(3A) | 0 ± 0 | 0 ± 0 | 0 ± 0 | 0 ± 0 | 0 ± 0 |
| SAg(2 μg) | | | | | |
| pSUPER-HBsAg-3(10 μg) + p(3A) | 2.8 ± 2.3 | 3.2 ± 2.5 | 1.4 ± 1.2 | 0.2 ± 0.8 | 0 ± 0 |
| SAg(5 μg) | | | | | |
| pSUPER-HBsAg-3(10 μg) + p(3A) | 43.4 ± 11.4 | 50.9 ± 11.1 | 27.9 ± 9.4 | 8.2 ± 3.9 | 2.1 ± 1.5 |
| SAg(10 μg) | | | | | |

The just-described experiments were repeated except that pHBV3.6, rather than p(3A)SAg, was used. Similar results were obtained. See Table 3 below.

TABLE 3
Effects of pSUPER-HBsAg-3 on pHBV3.6 directed HBsAg expression

| | Day after injection, HBsAg (ng/ml) | | | | |
|---|---|---|---|---|---|
| Treatment | Day 1 | Day 2 | Day 3 | Day 4 | Day 5 |
| pSUPER + pHBV3.6 | 426.70 ± 79.46 | 949.10 ± 177.80 | 1051.00 ± 170.00 | 556.10 ± 56.68 | 226.80 ± 16.54 |
| pSUPER-HBsAg-3 + pHBV3.6 | 0 | 0 | 0 | 0 | 0 |

Immunohistochemical staining was carried out to exam HBsAg expression in hepatocytes of the mice injected with pSUPER-HBsAg-3 and pHBV3.6. More specifically, mouse livers were embedded within O.C.T compound (Miles Inc., Elkhart, Ind.) and frozen in liquid nitrogen at Day 2 after hydrodynamics-based transfection of p(3A)SAg or pHBV3.6 and the siRNA construct. Four-micrometer cryosections were made using a cryostat (Leica CM 1800, Nussloch, Germany). After the sections were fixed in cold acetone, endogenous peroxidase was inhibited by 3% H₂O₂/PBS. The sections were then sequentially incubated with sheep polyclonal anti-HBs antibodies (Serotec, Oxford, UK), biotinylated anti-sheep secondary antibodies, and peroxidase-conjugated streptoavidin (DAKO, Carpinteria, Calif.). The HBsAg expression and cellular structure were visualized by incubating with aminoethyl carbazole (AEC, Zymed Laboratories, San Francisco, Calif.) and counterstaining with Mayer's hematoxylin (Merck, Darmstadt, Germany). It was found that injection pSUPER-HBsAg-3 repressed the hepatocyte expression of HBsAg.

HBV transcriptional intermediated fragments were analyzed by Northern blot to investigate the mechanism of pSUPER-HBsAg-3-induced inhibition of pHBV3.6. Thirty micrograms of total RNA were purified from the mouse liver using a TRI Reagent kit (Molecular Research Center, INC. Cincinnati, Ohio) 2 days after co-injection of HBV3.6 and pSUPER-HBsAg-3. The total RNA was subjected to Northern blot in the manner described above. It was shown that the levels of three HBV RNA fragments (3.5, 2.4 and 2.1 Kb) in the mice injected with pSUPER-HBsAg-3 were about 67% of those in the mice injected with the control vector pSUPER.

The serum HBV-DNA level in the injected mice was also measured. Mouse sera were collected at day 2 after the injection. The sera were treated with DNaseI (20U, >12 hours) to eliminate plasmid DNA. HBV DNA was then purified from 200 μl mouse serum using a viral DNA/RNA isolation kit (Maxim Biotech, INC., San Francisco, Calif.). Five microliters of the isolated DNA was subjected to a PCR analysis. The primers SP2-d1 (5′-GCGGGTCACCATATTCTTGG-3′) and SP2-d4 (5′-GAGTCTAGACTCTGCGGTAT-3′) were used to amplify the preS2 region of the surface antigen gene. It was found that serum HBV-DNA was completely inhibited by pSUPER-HBsAg-3. These results indicate that the RNAs transcribed from the RNAi vector inhibit the replication of the whole HBV genome.

To examine the level of HBV virion, serum anti-HBcAb level was used to represent the level of HBcAg synthesis. Anti-HBcAb was detected using a competition ELISA kit (General Biological Corp., Taiwan, R.O.C.) according to the manufacture's direction. The cutoff value of anti-HBcAb assay was calculated as 0.261 using the following formula: [(0.4×(A490 of negative control)+0.6×(A490 of positive control)]. The values below the cutoff value were defined as anti-HBcAg positive. The results are summarized in Table 4 below.

TABLE 4
Effects of pSUPER-HBsAg-3 on serum anti-HBcAg level

| | Effects on anti-HBcAg levels at various days after injection | | | |
|---|---|---|---|---|
| Treatment | Day 5 | Day 6 | Day 7 | Day 8 |
| pSUPER + pHBV3.6 | 0.375 ± 0.138 | 0.139 ± 0.025 | 0.132 ± 0.018 | 0.186 ± 0.035 |
| pSUPER-HBsAg-3 + pHBV3.6 | 0.556 ± 0.182 | 0.466 ± 0.282 | 0.425 ± 0.245 | 0.543 ± 0.180 |

As shown in Table 4, the anti-HBcAb levels were lower in the mice injected with pSUPER-HBsAg-3 than those in the mice injected with pSUPER. These results indicate that an HBsAg-specific siRNA-containing vector, such as pSUPER-HBsAg-3, inhibits HBsAg synthesis or HBV replication in vivo and that the vector has a preventive effect against HBV.

To investigate the therapeutic effect of pSUPER-HBsAg-3, 6 BALB/c mice were divided into two groups (3 in each) and were administrated intravenously with 5 μg of p(3A)SAg in the manner described above. Two days later, the mice were injected with 25 μg of pSUPER and pSUPER-HBsAg-3, respectively. The serum HBsAg level in each mouse was detected by ELISA before (“Day 0”) and after the second injection (“Days 1-5”). The results are summarized in Table 5 below.

TABLE 5
Therapeutic effects pSUPER-HBsAg-3 on p(3A)SAg

| | Serum HBsAg level (ng/ml) | | | | | |
|---|---|---|---|---|---|---|
| Treatment | Day 0 | Day 1 | Day 2 | Day 3 | Day 4 | Day 5 |
| p(3A)SAg + pSUPER | 439.05 ± 55.25 | 591.50 ± 19.87 | 426.20 ± 11.42 | 392.60 ± 85.68 | 316.70 ± 5.84 | 142.1 ± 18.24 |
| p(3A)SAg + pSUPER-HBsAg-3 | 439.05 ± 55.25 | 484.60 ± 97.79 | 210.30 ± 13.05 | 145.90 ± 17.71 | 103.30 ± 13.70 | 35.46 ± 2.23 |

As shown in Table 5, in the mice injected with p(3A)SAg and pSUPER-HBsAg-3, the average serum HBsAg level was inhibited by about 18% at day 1, 60% at days 2-4, and 75% at day 5 after the second injection. The experiment was repeated, except that pHBV3.6 was used. Similar results were obtained. The serum anti-HBcAg level was also measured in the manner described above and was found to be repressed in the mice injected with pSUPER-HBsAg-3. These results suggest that pSUPER-HBsAg-3 has therapeutic inhibition on HBsAg synthesis and HBV replication.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. An RNA for inhibiting expression of a gene of a virus, the RNA comprising a first nucleotide sequence that hybridizes under stringent conditions to a segment of the gene, and a second nucleotide sequence that is complementary to the first nucleotide sequence and hybridizes to the first nucleotide sequence to form a duplex structure.

2. The RNA of claim 1, wherein the first nucleotide sequence and the second nucleotide sequence are on the same strand.

3. The RNA of claim 1, wherein the RNA is a double-stranded RNA.

4. The RNA of claim 1, wherein the first nucleotide sequence is at least 19 nucleotides in length.

5. The RNA of claim 4, wherein the first nucleotide sequence is 19 to 29 nucleotides in length.

6. The RNA of claim 5, wherein the virus is a hepatitis virus.

7. The RNA of claim 6, wherein the virus is a hepatitis B virus.

8. The RNA of claim 7, wherein the segment contains one of SEQ ID NOs: 1-10.

9. The RNA of claim 8, wherein the segment contains SEQ ID NO: 3.

10. The RNA of claim 9, wherein the first nucleotide sequence and the second nucleotide sequence are on the same strand.

11. The RNA of claim 1, wherein the virus is a hepatitis virus.

12. The RNA of claim 11, wherein the virus is a hepatitis B virus.

13. The RNA of claim 12, wherein the segment contains one of SEQ ID NOs: 1-10.

14. The RNA of claim 13, wherein the segment contains SEQ ID NO: 3.

15. The RNA of claim 14, wherein the first nucleotide sequence and the second nucleotide sequence are on the same strand.

16. A DNA vector comprising a nucleic acid that encodes the RNA of claim 1.

17. The DNA vector of claim 16, wherein the first nucleotide sequence is at least 19 nucleotides in length.

18. The DNA vector of claim 17, wherein the first nucleotide sequence is 19 to 29 nucleotides in length.

19. The DNA vector of claim 18, wherein the virus is a hepatitis virus.

20. The DNA vector of claim 19, wherein the virus is a hepatitis B virus.

21. The DNA vector of claim 20, wherein the segment contains one of SEQ ID NOs: 1-10.

22. The DNA vector of claim 21, wherein the segment contains SEQ ID NO: 3.

23. The DNA vector of claim 16, wherein the virus is a hepatitis virus.

24. The DNA vector of claim 23, wherein the virus is a hepatitis B virus.

25. The DNA vector of claim 24, wherein the segment contains one of SEQ ID NOs: 1-10.

26. The DNA vector of claim 25, wherein the segment contains SEQ ID NO: 3.

27. A method of reducing the expression of a gene of a virus in a cell, the method comprising introducing into the cell an effective amount of the RNA of claim 1 or a DNA vector containing a nucleic acid encoding the RNA.

28. The method of claim 27, wherein the virus is a hepatitis virus.

29. The method of claim 28, wherein the virus is a hepatitis B virus.

30. The method of claim 29, wherein the segment contains one of SEQ ID NOs: 1-10.

31. The method of claim 30, wherein the segment contains SEQ ID NO: 3.

32. A method of inhibiting the replication of a virus in a cell, the method comprising introducing into the cell an effective amount of the RNA of claim 1 or a DNA vector containing a nucleic acid encoding the RNA.

33. The method of claim 32, wherein the virus is a hepatitis virus.

34. The method of claim 33, wherein the virus is a hepatitis B virus.

35. The method of claim 34, wherein the segment contains one of SEQ ID NOs: 1-10.

36. The method of claim 35, wherein the segment contains SEQ ID NO: 3.

37. A method of treating an infection with a virus, the method comprising administering to a subject in need thereof an effective amount of the RNA of claim 1 or a DNA vector containing a nucleic acid encoding the RNA.

38. The method of claim 37, wherein the virus is a hepatitis virus.

39. The method of claim 38, wherein the virus is a hepatitis B virus.

40. The method of claim 39, wherein the segment contains one of SEQ ID NOs: 1-10.

41. The method of claim 40, wherein the segment contains SEQ ID NO: 3.

42. A pharmaceutical composition comprising the RNA of claim 1 or a DNA vector containing a nucleic acid encoding the RNA, and a pharmaceutically acceptable carrier.


SEQUENCE LISTING (16 sequences)

SEQ ID NO: 1 - DNA, 19 bp, Hepatitis B virus
cctgcacgactcctgctca

SEQ ID NO: 2 - DNA, 19 bp, Hepatitis B virus
tacctatgggagtgggcct

SEQ ID NO: 3 - DNA, 19 bp, Hepatitis B virus
ggtatgttgcccgtttgtc

SEQ ID NO: 4 - DNA, 19 bp, Hepatitis B virus
ggcagcctactcccatctc

SEQ ID NO: 5 - DNA, 19 bp, Hepatitis B virus
gagacagtcatcctcaggc

SEQ ID NO: 6 - DNA, 19 bp, Hepatitis B virus
tctccgcgaggactgggga

SEQ ID NO: 7 - DNA, 19 bp, Hepatitis B virus
caaccagtacgggaccatg

SEQ ID NO: 8 - DNA, 19 bp, Hepatitis B virus
gtcctctaattccaggatc

SEQ ID NO: 9 - DNA, 19 bp, Hepatitis B virus
gcctcatcttcttattggt

SEQ ID NO: 10 - DNA, 19 bp, Hepatitis B virus
cctccaatcactcaccaac

SEQ ID NO: 11 - DNA, 64 bp, Artificial Sequence
  Synthetically generated oligonucleotide
gatccccnnnnnnnnnnnnnnnnnnnttcaagagannnnnnnnnnnnnnnnnnntttttggaaa

SEQ ID NO: 12 - DNA, 64 bp, Artificial Sequence
  Synthetically generated oligonucleotide
agcttttccaaaaannnnnnnnnnnnnnnnnnntctcttgaannnnnnnnnnnnnnnnnnnggg

SEQ ID NO: 13 - DNA, 61 bp, Artificial Sequence
  Synthetically generated oligonucleotide
cgnnnnnnnnnnnnnnnnnnnttcaagagannnnnnnnnnnnnnnnnnncttttttggaaa

SEQ ID NO: 14 - DNA, 63 bp, Artificial Sequence
  Synthetically generated oligonucleotide
agcttttccaaaaaagnnnnnnnnnnnnnnnnnnntctcttgaannnnnnnnnnnnnnnnnnn

SEQ ID NO: 15 - DNA, 20 bp, Artificial Sequence
  Primer
gcgggtcaccatattcttgg

SEQ ID NO: 16 - DNA, 20 bp, Artificial Sequence
  Primer
gagtctagactctgcggtat